(19) United States
(12) Patent Application Publication
(10) Pub. No.: US 2018/0220650 A1
(43) Pub. Date: Aug. 9, 2018

(54) PLANT GROWTH REGULATOR COMPOUNDS

(71) Applicant: Syngenta Participations AG, Basel (CH)

(72) Inventors: Alexandre Franco Jean Camille Lumbroso, Stein (CH); Alain De Mesmaeker, Stein (CH)

(73) Assignee: Syngenta Participations AG, Basel (CH)

(21) Appl. No.: 15/750,274

(22) PCT Filed: Aug. 4, 2016
(86) PCT No.: PCT/EP2016/068641

§ 371 Date: Feb. 5, 2018

(30) Foreign Application Priority Data
Aug. 7, 2015 (GB) 1513971.0

(51) Int. Cl. A01N 43/12; C07D 307/92; C07D 405/12; A01N 43/36

(52) U.S. Cl. CPC A01N 43/12; A01N 43/36; C07D 405/12; C07D 307/92

(57) ABSTRACT

The present invention relates to novel strigolactam derivatives, to processes for preparing these derivatives including intermediate compounds, to plant growth regulator or seed germination promoting compositions comprising these derivatives and to methods of using these derivatives in controlling the growth of plants and/or promoting the germination of seeds.

The present invention relates to novel strigolactam derivatives, to processes for preparing these derivatives including intermediate compounds, to seeds comprising these derivatives, to plant growth regulator or seed germination promoting compositions comprising these derivatives and to methods of using these derivatives in controlling the growth of plants and/or promoting the germination of seeds.

Strigolactone derivatives are phytohormones which may have plant growth regulation and seed germination properties. They have previously been described in the literature. Certain known strigolactam derivatives (eg, see WO 2012/080115 and WO 2015/061764) may have properties analogous to strigolactones, eg, plant growth regulation and/or seed germination promotion. Specifically, WO 2015/061764 discloses plant propagation materials comprising chemical mimics of strigolactone thought to be particularly effective under drought stress conditions.

For such compounds to be used, in particular, in seed treatment applications (eg, as seed coating components), hydrolytic stability and soil stability are important once a seed has been planted in the field in terms of maintaining the compound's biological activity.

According to the present invention, there is provided a compound of Formula (I):

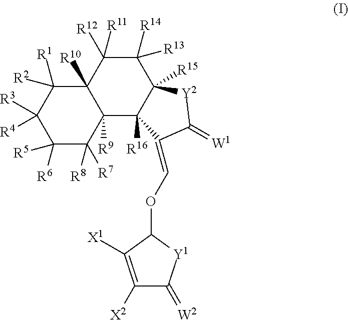

wherein

R¹, R², R³, R⁴, R⁵, R⁶, R⁷, R⁸, R⁹, R¹⁰, R¹¹, R¹², R¹³, R¹⁴ R¹⁵ and R¹⁶ are each independently hydrogen, C₁-C₆alkyl, C₁-C₆haloalkyl, halogen, OR¹⁷, cyano, or N(R¹⁸)₂, wherein R¹⁸ may the same or different;

R¹⁷ is hydrogen, C₁-C₆alkyl, C₁-C₆haloalkyl, C₂-C₆alkenyl, C₂-C₆alkynyl, C₃-C₆cycloalkyl, C₁-C₈alkylcarbonyl, C₁-C₈alkoxycarbonyl, substituted or unsubstituted aryl, substituted or unsubstituted heteroaryl, substituted or unsubstituted heterocyclyl, substituted or unsubstituted benzyl;

R¹⁸ is hydrogen, C₁-C₆alkyl, C₁-C₆alkoxy, C₃-C₆cycloalkyl, C₂-C₆alkenyl, C₂-C₆alkynyl, C₁-C₈alkylcarbonyl, C₁-C₈alkoxycarbonyl, hydroxyl, amino, N—C₁-C₆alkylamine, N,N-di-C₁-C₆alkylamine, substituted or unsubstituted aryl, substituted or unsubstituted heteroaryl, substituted or unsubstituted heterocyclyl, substituted or unsubstituted benzyl;

W¹ and W² are independently oxygen or sulfur;

Y¹ and Y² are independently oxygen, sulfur, or NR¹⁹;

R¹⁹ is hydrogen, C₁-C₆alkyl, C₁-C₆haloalkyl, C₁-C₆alkoxy, C₃-C₆cycloalkyl, C₂-C₆alkenyl, C₂-C₆alkynyl, C₁-C₈alkylcarbonyl, C₁-C₈alkoxycarbonyl, hydroxyl, amine, N—C₁-C₆alkylamine, N,N-di-C₁-C₆alkylamine, substituted or unsubstituted aryl, substituted or unsubstituted heteroaryl, substituted or unsubstituted heterocyclyl, substituted or unsubstituted benzyl; and

X¹ is selected from C₁-C₆alkyl, C₁-C₆haloalkyl, C₂-C₃alkynyl, halogen, hydroxyl, C₁-C₆alkoxy, C₁-C₆alkylsulfinyl, C₁-C₆alkylsulfonyl, C₁-C₆alkylthio, OR¹⁷ and N(R¹⁸)₂;

X² is selected from hydrogen, C₁-C₆alkyl, C₁-C₆haloalkyl, C₂-C₃alkynyl, halogen, hydroxyl, C₁-C₆alkoxy, C₁-C₆alkylsulfinyl, C₁-C₆alkylsulfonyl, C₁-C₆alkylthio, OR¹⁷ and N(R¹⁸)₂; or

X¹ and X² together with the carbon atoms to which they are attached form a C₅- or C₆-cycloalkyl;

or salts or N-oxides thereof.

The compounds of Formula (I) may exist in different geometric or optical isomers (diastereoisomers and enantiomers) or tautomeric forms. This invention covers all such isomers and tautomers and mixtures thereof in all proportions as well as isotopic forms such as deuterated compounds. The invention also covers all salts, N-oxides, and metalloidic complexes of the compounds of Formula (I).

Each alkyl moiety either alone or as part of a larger group (such as alkoxy, alkoxycarbonyl, alkylcarbonyl, alkylaminocarbonyl, dialkylaminocarbonyl) is a straight or branched chain and is, for example, but not limited to, methyl, ethyl, n-propyl, n-butyl, n-pentyl, n-hexyl, isopropyl, sec-butyl, isobutyl, tert-butyl or neo-pentyl. The alkyl groups include C₁-C₆alkyl, C₁-C₄alkyl, and C₁-C₃alkyl.

The term “alkenyl”, as used herein, is an alkyl moiety having at least one carbon-carbon double bond, for example C₂-C₆alkenyl. Specific examples include vinyl and allyl. The alkenyl moiety may be part of a larger group (such as alkenoxy, alkenoxycarbonyl, alkenylcarbonyl, alkyenlaminocarbonyl, dialkenylaminocarbonyl).

The term “alkynyl”, as used herein, is an alkyl moiety having at least one carbon-carbon triple bond, for example C₂-C₆alkynyl. Specific examples include ethynyl and propargyl. The alkynyl moiety may be part of a larger group (such as alkynoxy, alkynoxycarbonyl, alkynylcarbonyl, alkynylaminocarbonyl, dialkynylaminocarbonyl).

Unless otherwise indicated, alkenyl and alkynyl, on their own or as part of another substituent, may be straight or branched chain, and where appropriate, may be in either the (E)- or (Z)-configuration. Examples include vinyl, allyl, ethynyl and propargyl.

Halogen (or halo) encompasses fluorine (F), chlorine (Cl), bromine (Br) or iodine (I). The same correspondingly applies to halogen in the context of other definitions, such as haloalkyl or halophenyl.

Haloalkyl groups (either alone or as part of a larger group, such as haloalkoxy or haloalkylthio) are alkyl groups which are substituted with one or more of the same or different halogen atoms and are, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl, heptafluoro-n-propyl and perfluoro-n-hexyl.

The term “nitro” refers to a radical of the formula —NO₂.

The term “hydroxyl” refers to a radical of the formula —OH.

The term “cyano” refers to a radical of the formula —C≡N.

Hydroxyalkyl groups are alkyl groups which are substituted with one or more hydroxyl group and are, for example, —CH₂OH, —CH₂CH₂OH or —CH(OH)CH₃.

Alkoxy groups are alkyl groups singular bonded to oxygen (—OR). Examples of alkoxy groups are, but are not limited to, methoxy, ethoxy, propoxy, isopropoxy, n-butoxy, isobutoxy, sec-butoxy or tert-butoxy or a pentyloxy or hexyloxy isomer. It should also be appreciated that two alkoxy substituents may be present on the same carbon atom.

The term “alkylthio” refers to a radical of the formula C₁-C₆alkyl-S—, and is, for example, but not limited to, methylthio, ethylthio, propylthio, isopropylthio, n-butylthio, isobutylthio, sec-butylthio or tert-butylthio.

The term “alkylsulfinyl” refers to a radical of the formula C₁-C₆alkyl-S(O)—, and is, for example, but not limited to, methylsulfinyl, ethylsulfinyl, propylsulfinyl, isopropylsulfinyl, n-butylsulfinyl, isobutyl-sulfinyl, sec-butylsulfinyl or tert-butylsulfinyl.

The term “alkylsulfonyl” refers to a radical of the formula C₁-C₆alkyl-S(O)₂—, and is, for example, but not limited to, methylsulfonyl, ethylsulfonyl, propylsulfonyl, isopropylsulfonyl, n-butylsulfonyl, isobutylsulfonyl, sec-butylsulfonyl or tert-butylsulfonyl.

Alkoxyalkyl groups are an alkoxy group bonded to an alkyl (R—O—R′), for example —(CH₂)ᵣO(CH₂)ₛCH₃, wherein r is 1 to 6 and s is 1 to 5.

In the context of the present specification the term “aryl” refers to an optionally substituted aromatic ring system which may be mono-, bi- or tricyclic, with 6 to 14 members. Examples of such rings include, but are not limited to, phenyl, benzyl, naphthalenyl, anthracenyl, indenyl or phenanthrenyl.

Unless otherwise indicated, the term “cycloalkyl” refers to a non-aromatic monocyclic or polycyclic ring comprising carbon and hydrogen, having from 3 to 7 members per ring, and may be optionally substituted by one or more C₁-C₆alkyl groups. Examples of cycloalkyl include, but are not limited to, cyclopropyl, 1-methylcyclopropyl, 2-methylcyclopropyl, cyclobutyl, cyclopentyl and cyclohexyl.

The term “heterocyclyl” refers to a ring system containing at least one heteroatom, and includes heteroaryl, saturated analogues, and in addition their unsaturated or partially unsaturated analogues such as 4,5,6,7-tetrahydro-benzothiophenyl, 9H-fluorenyl, 3,4-dihydro-2H-benzo-1,4-dioxepinyl, 2,3-dihydro-benzofuranyl, piperidinyl, 1,3-dioxolanyl, 1,3-dioxanyl, 4,5-dihydro-isoxazolyl, tetrahydrofuranyl and morpholinyl. In addition, the term “heterocyclyl” includes heterocycloalkyl, a non-aromatic monocyclic or polycyclic ring comprising carbon and hydrogen atoms and at least one heteroatom selected from nitrogen, oxygen, and sulfur such asoxetanyl or thietanyl.

The term “heteroaryl” refers to an aromatic ring system having from 3 to 9 members per ring, containing at least one heteroatom and consisting either of a single ring or of two or more fused rings. Single rings may contain up to three heteroatoms, and bicyclic systems up to four heteroatoms, which will preferably be chosen from nitrogen, oxygen and sulfur. Examples of such groups include pyridyl, pyridazinyl, pyrimidinyl, pyrazinyl, furanyl, thiophenyl, oxazolyl, isoxazolyl, oxadiazolyl, thiazolyl, isothiazolyl, thiadiazolyl, pyrrolyl, pyrazolyl, imidazolyl, triazolyl and tetrazolyl.

The term “alkylcarbonyl” refers to a radical of the formula —C(═O)—Ra where Ra is an alkyl radical as defined above. Examples of alkylcarbonyl include, but are not limited to, acetyl.

The term “alkoxycarbonyl” refers to a radical of the formula —C(═O)—O—Ra, where Ra is an alkyl radical as defined above. Examples of C₁-C₆alkoxycarbonyl include, but are not limited to, methoxycarbonyl, ethoxycarbonyl and isopropoxycarbonyl.

The term “N-alkylamine” refers to a radical of the formula —NH—Ra where Ra is an alkyl radical as defined above.

The term “N,N-dialkylamino” refers to a radical of the formula —N(Ra)—Ra where each Ra is an alkyl radical, which may be the same or different, as defined above.

The term “benzyl” refers to a —CH₂C₆H₅ radical.

Preferred values of W¹, W², Y¹, Y², X¹, X², R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹ and R²⁰ are, in any combination, as set out below:

In one embodiment, W¹ is oxygen.

In a second embodiment W¹ is sulfur.

In one embodiment, W² is oxygen.

In a second embodiment W² is sulfur.

Preferably, W¹ and W² are both oxygen.

R¹, R², R³, R⁴, R⁵, R⁶, R⁷, R⁸, R⁹, R¹⁰, R¹¹, R¹², R¹³, and R¹⁴ are preferably independently selected from hydrogen, C₁-C₆alkyl, C₁-C₆haloalkyl, and halogen. In one embodiment, R¹, R², R³, R⁴, R⁵, R⁶, R⁷, R⁸, R⁹, R¹⁰, R¹¹, R¹², R¹³, and R¹⁴ are independently selected from hydrogen, halogen, methyl, ethyl and tert-butyl.

R¹ and R² are preferably independently selected from hydrogen, halogen and C₁-C₃alkyl. In one embodiment, R¹ and R² are methyl.

R³ and R⁴ are preferably independently selected from hydrogen, halogen and C₁-C₃alkyl. In one embodiment, R³ and R⁴ are independently selected from halogen and methyl. In another embodiment, R³ and R⁴ are hydrogen.

R⁵ and R⁶ are preferably independently selected from hydrogen, halogen and C₁-C₃alkyl. In one embodiment, R⁵ and R⁶ are independently selected from halogen and methyl. In another embodiment, R⁵ and R⁶ are hydrogen.

R⁷ and R⁸ are preferably independently selected from hydrogen, halogen and C₁-C₃alkyl. In one embodiment, R⁷ and R⁸ are independently selected from halogen and methyl. In another embodiment R⁷ and R⁸ are hydrogen.

R⁹ is preferably hydrogen or C₁-C₃alkyl. In one embodiment, R⁹ is methyl. In another embodiment, R⁹ is hydrogen.

R¹⁰ is preferably hydrogen or C₁-C₃alkyl. In one embodiment, R¹⁰ is hydrogen. In another embodiment, R¹⁰ is methyl.

R¹¹ and R¹² are preferably independently selected from hydrogen, halogen and C₁-C₃alkyl. In one embodiment, R¹¹ and R¹² are independently selected from halogen and methyl. In another embodiment, R¹¹ and R¹² are hydrogen.

R¹³ and R¹⁴ are preferably independently selected from hydrogen, halogen and C₁-C₃alkyl. In one embodiment, R¹³ and R¹⁴ are independently selected from halogen and methyl. In another embodiment, R¹³ and R¹⁴ are hydrogen.

R¹⁵ is preferably hydrogen or C₁-C₃alkyl. In one embodiment, R¹⁵ is methyl. In another embodiment R¹⁵ is hydrogen.

R¹⁶ is preferably hydrogen or C₁-C₃alkyl. In one embodiment, R¹⁶ is hydrogen. In another embodiment R¹⁶ is methyl.

Preferably R¹⁷ is hydrogen or C₁-C₆alkyl. In one embodiment R¹⁷ is hydrogen, methyl, ethyl, isopropyl or tert-butyl. In another embodiment R¹⁷ is hydrogen or methyl.

Preferably R¹⁸ is hydrogen, C₁-C₆alkyl, C₁-C₆haloalkyl, C₁-C₆alkoxy, or substituted or unsubstituted aryl. In one embodiment R¹⁸ is hydrogen or C₁-C₃alkyl. In another embodiment R¹⁸ is hydrogen or methyl.

In one embodiment Y¹ is oxygen. In a second embodiment Y¹ is —N(R¹⁹).

Preferably R¹⁹ is hydrogen, C₁-C₃alkoxy, C₁-C₃haloalkyl, C₃-C₆cycloalkyl, substituted aryl or unsubstituted aryl. In one embodiment R¹⁹ is substituted aryl or unsubstituted aryl. In a second embodiment R¹⁹ is phenyl or phenyl substituted by one to five R²⁰, wherein each R²⁰ is independently C₁-C₄alkyl, C₁-C₄haloalkyl, C₁-C₄alkoxy, or C₁-C₄haloalkoxy. In another embodiment R¹⁹ is phenyl or halo-substituted phenyl. In a further embodiment R¹⁹ is phenyl or 3,5-bis(trifluoromethyl)phenyl. In an additional embodiment R¹⁹ is phenyl.

Preferably Y² is oxygen.

Surprisingly we have found that when X¹ is not hydrogen the compounds of the present invention exhibit greater stability.

Preferably X¹ is selected from C₁-C₆alkyl, C₁-C₆haloalkyl, halogen, hydroxyl, and C₁-C₆alkoxy.

In one embodiment X¹ is methyl, ethyl, n-propyl, isopropyl, methoxy, ethoxy, n-propoxy, or isopropoxy. In another embodiment X¹ is methyl or methoxy. In a further embodiment X¹ is methyl.

Preferably X² is selected from hydrogen, C₁-C₆alkyl, C₁-C₆haloalkyl, halogen, hydroxyl, and C₁-C₆alkoxy. In one embodiment X² is methyl, ethyl, n-propyl, isopropyl, methoxy, ethoxy, n-propoxy, or isopropoxy. In another embodiment X² is methyl or methoxy. In a further embodiment X² is methyl.

In one embodiment of Formula (I):

R¹, R², R³, R⁴, R⁵, R⁶, R⁷, R⁸, R⁹, R¹⁰, R¹¹, R¹², R¹³, R¹⁴ R¹⁵ and R¹⁶ are each independently hydrogen, C₁-C₆alkyl, C₁-C₆haloalkyl, halogen, OR¹⁷, cyano, or N(R¹⁸)₂, wherein R¹⁸ may the same or different;

R¹⁷ is hydrogen, C₁-C₆alkyl, C₁-C₆haloalkyl, C₂-C₆alkenyl, C₂-C₆alkynyl, C₃-C₆cycloalkyl, C₁-C₈alkylcarbonyl, C₁-C₈alkoxycarbonyl, substituted or unsubstituted aryl, substituted or unsubstituted heteroaryl, substituted or unsubstituted heterocyclyl, substituted or unsubstituted benzyl;

R¹⁸ is hydrogen, C₁-C₆alkyl, C₁-C₆alkoxy, C₃-C₆cycloalkyl, C₂-C₆alkenyl, C₂-C₆alkynyl, C₁-C₈alkylcarbonyl, C₁-C₈alkoxycarbonyl, hydroxyl, amino, N—C₁-C₆alkylamine, N,N-di-C₁-C₆alkylamine, substituted or unsubstituted aryl, substituted or unsubstituted heteroaryl, substituted or unsubstituted heterocyclyl, substituted or unsubstituted benzyl;

W¹ and W² are independently oxygen or sulfur;

Y¹ and Y² are independently oxygen, sulfur, or NR¹⁹;

R¹⁹ is hydrogen, C₁-C₆alkyl, C₁-C₆haloalkyl, C₁-C₆alkoxy, C₃-C₆cycloalkyl, C₂-C₆alkenyl, C₂-C₆alkynyl, C₁-C₈alkylcarbonyl, C₁-C₈alkoxycarbonyl, hydroxyl, amine, N—C₁-C₆alkylamine, N,N-di-C₁-C₆alkylamine, substituted or unsubstituted aryl, substituted or unsubstituted heteroaryl, substituted or unsubstituted heterocyclyl, substituted or unsubstituted benzyl; and

X¹ and X² are independently selected from methyl, ethyl and methoxy.

Preferred values of W¹, W², Y¹, Y², X¹, X², R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, and R¹⁹ are as set out above.

In a further embodiment of Formula (I):

R¹, R², R³, R⁴, R⁵, R⁶, R⁷, R⁸, R⁹, R¹⁰, R¹¹, R¹², R¹³, R¹⁴ R¹⁵ and R¹⁶ are each independently hydrogen, C₁-C₆alkyl, C₁-C₆haloalkyl, halogen, OR¹⁷, cyano, or N(R¹⁸)₂, wherein R¹⁸ may the same or different;

R¹⁷ is hydrogen, C₁-C₆alkyl, C₁-C₆haloalkyl, C₂-C₆alkenyl, C₂-C₆alkynyl, C₃-C₆cycloalkyl, C₁-C₈alkylcarbonyl, C₁-C₈alkoxycarbonyl, substituted or unsubstituted aryl, substituted or unsubstituted heteroaryl, substituted or unsubstituted heterocyclyl, substituted or unsubstituted benzyl; R¹⁸ is hydrogen, C₁-C₆alkyl, C₁-C₆alkoxy, C₃-C₆cycloalkyl, C₂-C₆alkenyl, C₂-C₆alkynyl, C₁-C₈alkylcarbonyl, C₁-C₈alkoxycarbonyl, hydroxyl, amino, N—C₁-C₆alkylamine, N,N-di-C₁-C₆alkylamine, substituted or unsubstituted aryl, substituted or unsubstituted heteroaryl, substituted or unsubstituted heterocyclyl, substituted or unsubstituted benzyl;

W¹ and W² are independently oxygen or sulfur;

Y¹ and Y² are independently oxygen, sulfur, or NR¹⁹;

R¹⁹ is hydrogen, C₁-C₆alkyl, C₁-C₆haloalkyl, C₁-C₆alkoxy, C₃-C₆cycloalkyl, C₂-C₆alkenyl, C₂-C₆alkynyl, C₁-C₈alkylcarbonyl, C₁-C₈alkoxycarbonyl, hydroxyl, amine, N—C₁-C₆alkylamine, N,N-di-C₁-C₆alkylamine, substituted or unsubstituted aryl, substituted or unsubstituted heteroaryl, substituted or unsubstituted heterocyclyl, substituted or unsubstituted benzyl; and

X¹ and X² are both methyl.

An example of this embodiment is a compound of Formula (Ia):

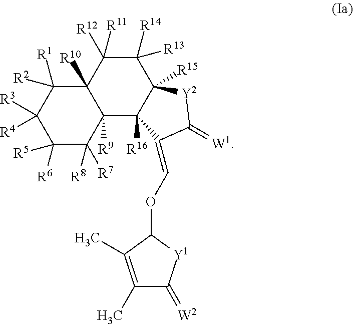

Preferred values of W¹, W², Y¹, Y², X¹, X², R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, and R¹⁹ are as set out above.

In another embodiment of Formula (I):

R¹, R², R⁹, and R¹⁵ are methyl;

R³, R⁴, R⁵, R⁶, R⁷, R⁸, R¹⁰, R¹¹, R¹², R¹³, R¹⁴ and R¹⁶ are hydrogen;

Y² and W¹ are oxygen;

W² is oxygen or sulfur;

Y¹ is oxygen, sulfur or NR¹⁹;

R¹⁹ is hydrogen, C₁-C₆alkyl, C₁-C₆haloalkyl, C₁-C₆alkoxy, C₃-C₆cycloalkyl, C₂-C₆alkenyl, C₂-C₆alkynyl, C₁-C₈alkylcarbonyl, C₁-C₈alkoxycarbonyl, hydroxyl, amine, N—C₁-C₆alkylamine, N,N-di-C₁-C₆alkylamine, substituted or unsubstituted aryl, substituted or unsubstituted heteroaryl, substituted or unsubstituted heterocyclyl, substituted or unsubstituted benzyl; and

X¹ is selected from C₁-C₆alkyl, C₂-C₃alkynyl, C₁-C₆haloalkyl, halogen, hydroxyl, C₁-C₆alkoxy, C₁-C₆alkylsulfinyl, C₁-C₆alkylsulfonyl, C₁-C₆alkylthio, OR¹⁷ and N(R¹⁸)₂;

X² is selected from hydrogen, C₁-C₆alkyl, C₂-C₃alkynyl, C₁-C₆haloalkyl, halogen, hydroxyl, C₁-C₆alkoxy, C₁-C₆alkylsulfinyl, C₁-C₆alkylsulfonyl, C₁-C₆alkylthio, OR¹⁷ and N(R¹⁸)₂; or

X¹ and X² together with the carbon atoms to which they are attached form a C₅- or C₆-cycloalkyl.

An example of this embodiment is a compound of Formula (Ib):

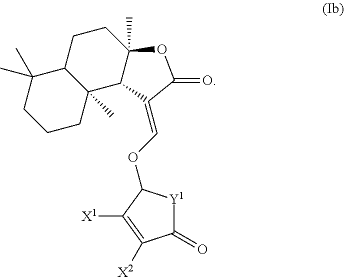

Preferred values of W¹, W², Y¹, Y², X¹, X², R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, and R¹⁹ are as set out above.

Table 1 below includes examples of compounds of the present invention.

TABLE 1
Compounds of Formula I
(I)

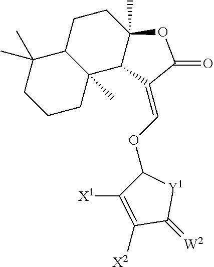

| Com- | | | | |
|---|---|---|---|---|
| pound | W² | Y¹ | X¹ | X² |
| Ib-1 | O | N—Ph | —CH₃ | —CH₃ |
| Ib-2 | O | N—Ph | —CH₃ | —C₂H₅ |
| Ib-3 | O | N—Ph | —CH₃ | —OCH₃ |
| Ib-4 | O | N—Ph | —C₂H₅ | —CH₃ |
| Ib-5 | O | N—Ph | —C₂H₅ | —C₂H₅ |
| Ib-6 | O | N—Ph | —C₂H₅ | —OCH₃ |
| Ib-7 | O | N—Ph | —OCH₃ | —CH₃ |
| Ib-8 | O | N—Ph | —OCH₃ | —C₂H₅ |
| Ib-9 | O | N—Ph | —OCH₃ | —OCH₃ |
| Ib-10 | O | N-C₆H₅(CF₃)₂ | —CH₃ | —CH₃ |
| Ib-11 | O | N-C₆H₅(CF₃)₂ | —CH₃ | —C₂H₅ |
| Ib-12 | O | N-C₆H₅(CF₃)₂ | —CH₃ | —OCH₃ |
| Ib-13 | O | N-C₆H₅(CF₃)₂ | —C₂H₅ | —CH₃ |
| Ib-14 | O | N-C₆H₅(CF₃)₂ | —C₂H₅ | —C₂H₅ |
| Ib-15 | O | N-C₆H₅(CF₃)₂ | —C₂H₅ | —OCH₃ |
| Ib-16 | O | N-C₆H₅(CF₃)₂ | —OCH₃ | —CH₃ |
| Ib-17 | O | N-C₆H₅(CF₃)₂ | —OCH₃ | —C₂H₅ |
| Ib-18 | O | N-C₆H₅(CF₃)₂ | —OCH₃ | —OCH₃ |
| Ib-19 | O | O | —CH₃ | —CH₃ |
| Ib-20 | O | O | —CH₃ | —C₂H₅ |
| Ib-21 | O | O | —CH₃ | —OCH₃ |
| Ib-22 | O | O | —C₂H₅ | —CH₃ |
| Ib-23 | O | O | —C₂H₅ | —C₂H₅ |
| Ib-24 | O | O | —C₂H₅ | —OCH₃ |
| Ib-25 | O | O | —OCH₃ | —CH₃ |
| Ib-26 | O | O | —OCH₃ | —C₂H₅ |
| Ib-27 | O | O | —OCH₃ | —OCH₃ |
| Ic-1 | S | N—Ph | —CH₃ | —CH₃ |
| Ic-2 | S | N—Ph | —CH₃ | —C₂H₅ |
| Ic-3 | S | N—Ph | —CH₃ | —OCH₃ |
| Ic-4 | S | N—Ph | —C₂H₅ | —CH₃ |
| Ic-5 | S | N—Ph | —C₂H₅ | —C₂H₅ |
| Ic-6 | S | N—Ph | —C₂H₅ | —OCH₃ |
| Ic-7 | S | N—Ph | —OCH₃ | —CH₃ |
| Ic-8 | S | N—Ph | —OCH₃ | —C₂H₅ |
| Ic-9 | S | N—Ph | —OCH₃ | —OCH₃ |
| Ic-10 | S | N-C₆H₅(CF₃)₂ | —CH₃ | —CH₃ |
| Ic-11 | S | N-C₆H₅(CF₃)₂ | —CH₃ | —C₂H₅ |
| Ic-12 | S | N-C₆H₅(CF₃)₂ | —CH₃ | —OCH₃ |
| Ic-13 | S | N-C₆H₅(CF₃)₂ | —C₂H₅ | —CH₃ |
| Ic-14 | S | N-C₆H₅(CF₃)₂ | —C₂H₅ | —C₂H₅ |
| Ic-15 | S | N-C₆H₅(CF₃)₂ | —C₂H₅ | —OCH₃ |
| Ic-16 | S | N-C₆H₅(CF₃)₂ | —OCH₃ | —CH₃ |
| Ic-17 | S | N-C₆H₅(CF₃)₂ | —OCH₃ | —C₂H₅ |
| Ic-18 | S | N-C₆H₅(CF₃)₂ | —OCH₃ | —OCH₃ |
| Ic-19 | S | O | —CH₃ | —CH₃ |
| Ic-20 | S | O | —CH₃ | —C₂H₅ |
| Ic-21 | S | O | —CH₃ | —OCH₃ |
| Ic-22 | S | O | —C₂H₅ | —CH₃ |
| Ic-23 | S | O | —C₂H₅ | —C₂H₅ |
| Ic-24 | S | O | —C₂H₅ | —OCH₃ |
| Ic-25 | S | O | —OCH₃ | —CH₃ |
| Ic-26 | S | O | —OCH₃ | —C₂H₅ |
| Ic-27 | S | O | —OCH₃ | —OCH₃ |

N-C₆H₅(CF₃)₂ = 3,5-bis(trifluoromethyl)phenyl

The present invention provides a method of improving the tolerance of a plant to abiotic stress, wherein the method comprises applying to the plant, plant part, plant propagation material, or plant growing locus a compound, composition or mixture according to the present invention.

The present invention provides a method for regulating or improving the growth of a plant, wherein the method comprises applying to the plant, plant part, plant propagation material, or plant growing locus a compound, composition or mixture according to the present invention. In one embodiment, plant growth is regulated or improved when the plant is subject to abiotic stress conditions.

The present invention also provides a method for improving seed germination of a plant, and especially the present invention provides a method for improving seed germination of a plant under cold stress conditions, comprising applying to the seed, or a locus containing seeds, a compound, composition or mixture according to the present invention.

The present invention also provides a method for safening a plant against phytotoxic effects of chemicals, comprising applying to the plant, plant part, plant propagation material, or plant growing locus a compound, composition or mixture according to the present invention.

Suitably the compound or composition is applied in an amount sufficient to elicit the desired response.

According to the present invention, “regulating or improving the growth of a crop” means an improvement in plant vigour, an improvement in plant quality, improved tolerance to stress factors, and/or improved input use efficiency.

An ‘improvement in plant vigour’ means that certain traits are improved qualitatively or quantitatively when compared with the same trait in a control plant which has been grown under the same conditions in the absence of the method of the invention. Such traits include, but are not limited to, early and/or improved germination, improved emergence, the ability to use fewer seeds, increased root growth, a more developed root system, increased root nodulation, increased shoot growth, increased tillering, stronger tillers, more productive tillers, increased or improved plant stand, less plant verse (lodging), an increase and/or improvement in plant height, an increase in plant weight (fresh or dry), bigger leaf blades, greener leaf colour, increased pigment content, increased photosynthetic activity, earlier flowering, longer panicles, early grain maturity, increased seed, fruit or pod size, increased pod or ear number, increased seed number per pod or ear, increased seed mass, enhanced seed filling, fewer dead basal leaves, delay of senescence, improved vitality of the plant, increased levels of amino acids in storage tissues and/or fewer inputs needed (e.g. less fertiliser, water and/or labour needed). A plant with improved vigour may have an increase in any of the aforementioned traits or any combination or two or more of the aforementioned traits.

An ‘improvement in plant quality’ means that certain traits are improved qualitatively or quantitatively when compared with the same trait in a control plant which has been grown under the same conditions in the absence of the method of the invention. Such traits include, but are not limited to, improved visual appearance of the plant, reduced ethylene (reduced production and/or inhibition of reception), improved quality of harvested material, e.g. seeds, fruits, leaves, vegetables (such improved quality may manifest as improved visual appearance of the harvested material), improved carbohydrate content (e.g. increased quantities of sugar and/or starch, improved sugar acid ratio, reduction of reducing sugars, increased rate of development of sugar), improved protein content, improved oil content and composition, improved nutritional value, reduction in anti-nutritional compounds, improved organoleptic properties (e.g. improved taste) and/or improved consumer health benefits (e.g. increased levels of vitamins and anti-oxidants), improved post-harvest characteristics (e.g. enhanced shelf-life and/or storage stability, easier processability, easier extraction of compounds), more homogenous crop development (e.g. synchronised germination, flowering and/or fruiting of plants), and/or improved seed quality (e.g. for use in following seasons). A plant with improved quality may have an increase in any of the aforementioned traits or any combination or two or more of the aforementioned traits.

An ‘improved tolerance to stress factors’ means that certain traits are improved qualitatively or quantitatively when compared with the same trait in a control plant which has been grown under the same conditions in the absence of the method of the invention. Such traits include, but are not limited to, an increased tolerance and/or resistance to abiotic stress factors which cause sub-optimal growing conditions such as drought (e.g. any stress which leads to a lack of water content in plants, a lack of water uptake potential or a reduction in the water supply to plants), cold exposure, heat exposure, osmotic stress, UV stress, flooding, increased salinity (e.g. in the soil), increased mineral exposure, ozone exposure, high light exposure and/or limited availability of nutrients (e.g. nitrogen and/or phosphorus nutrients). A plant with improved tolerance to stress factors may have an increase in any of the aforementioned traits or any combination or two or more of the aforementioned traits. In the case of drought and nutrient stress, such improved tolerances may be due to, for example, more efficient uptake, use or retention of water and nutrients.

In particular, the compounds or compositions of the present invention are useful to improve tolerance to drought stress.

An ‘improved input use efficiency’ means that the plants are able to grow more effectively using given levels of inputs compared to the growth of control plants which are grown under the same conditions in the absence of the method of the invention. In particular, the inputs include, but are not limited to fertiliser (such as nitrogen, phosphorous, potassium, and micronutrients), light and water. A plant with improved input use efficiency may have an improved use of any of the aforementioned inputs or any combination of two or more of the aforementioned inputs.

Other effects of regulating or improving the growth of a crop include a decrease in plant height, or reduction in tillering, which are beneficial features in crops or conditions where it is desirable to have less biomass and fewer tillers.

Any or all of the above crop enhancements may lead to an improved yield by improving e.g. plant physiology, plant growth and development and/or plant architecture. In the context of the present invention ‘yield’ includes, but is not limited to, (i) an increase in biomass production, grain yield, starch content, oil content and/or protein content, which may result from (a) an increase in the amount produced by the plant per se or (b) an improved ability to harvest plant matter, (ii) an improvement in the composition of the harvested material (e.g. improved sugar acid ratios, improved oil composition, increased nutritional value, reduction of anti-nutritional compounds, increased consumer health benefits) and/or (iii) an increased/facilitated ability to harvest the crop, improved processability of the crop and/or better storage stability/shelf life. Increased yield of an agricultural plant means that, where it is possible to take a quantitative measurement, the yield of a product of the respective plant is increased by a measurable amount over the yield of the same product of the plant produced under the same conditions, but without application of the present invention. According to the present invention, it is preferred that the yield be increased by at least 0.5%, more preferred at least 1%, even more preferred at least 2%, still more preferred at least 4%, preferably 5% or even more.

Any or all of the above crop enhancements may also lead to an improved utilisation of land, i.e. land which was previously unavailable or sub-optimal for cultivation may become available. For example, plants which show an increased ability to survive in drought conditions, may be able to be cultivated in areas of sub-optimal rainfall, e.g. perhaps on the fringe of a desert or even the desert itself.

In one aspect of the present invention, crop enhancements are made in the substantial absence of pressure from pests and/or diseases and/or abiotic stress. In a further aspect of the present invention, improvements in plant vigour, stress tolerance, quality and/or yield are made in the substantial absence of pressure from pests and/or diseases. For example pests and/or diseases may be controlled by a pesticidal treatment that is applied prior to, or at the same time as, the method of the present invention. In a still further aspect of the present invention, improvements in plant vigour, stress tolerance, quality and/or yield are made in the absence of pest and/or disease pressure. In a further embodiment, improvements in plant vigour, quality and/or yield are made in the absence, or substantial absence, of abiotic stress.

The compounds of the present invention can be used alone, but are generally formulated into compositions using formulation adjuvants, such as carriers, solvents and surface-active agents (SFAs). Thus, the present invention further provides a composition comprising a compound of the present invention and an agriculturally acceptable formulation adjuvant. There is also provided a composition consisting essentially of a compound of the present invention and an agriculturally acceptable formulation adjuvant. There is also provided a composition consisting of a compound of the present invention and an agriculturally acceptable formulation adjuvant.

The present invention further provides a plant growth regulator composition comprising a compound of the present invention and an agriculturally acceptable formulation adjuvant. There is also provided a plant growth regulator composition consisting essentially of a compound of the present invention and an agriculturally acceptable formulation adjuvant. There is also provided a plant growth regulator composition consisting of a compound of the present invention and an agriculturally acceptable formulation adjuvant.

The present invention further provides a plant abiotic stress management composition comprising a compound of the present invention and an agriculturally acceptable formulation adjuvant. There is also provided a plant abiotic stress management composition consisting essentially of a compound of the present invention and an agriculturally acceptable formulation adjuvant. There is also provided a plant abiotic stress management composition consisting of a compound of the present invention and an agriculturally acceptable formulation adjuvant.

The present invention further provides a seed germination promoter composition comprising a compound of the present invention and an agriculturally acceptable formulation adjuvant. There is also provided a seed germination promoter composition consisting essentially of a compound of the present invention and an agriculturally acceptable formulation adjuvant. There is also provided a seed germination promoter composition consisting of a compound of the present invention and an agriculturally acceptable formulation adjuvant.

The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The compositions generally comprise from 0.1 to 99% by weight, especially from 0.1 to 95% by weight, compounds of the present invention are from 1 to 99.9% by weight of a formulation adjuvant which preferably includes from 0 to 25% by weight of a surface-active substance.

The compositions can be chosen from a number of formulation types, many of which are known from the Manual on Development and Use of FAO Specifications for Plant Protection Products, 5th Edition, 1999. These include dustable powders (DP), soluble powders (SP), water soluble granules (SG), water dispersible granules (WG), wettable powders (WP), granules (GR) (slow or fast release), soluble concentrates (SL), oil miscible liquids (OL), ultralow volume liquids (UL), emulsifiable concentrates (EC), dispersible concentrates (DC), emulsions (both oil in water (EW) and water in oil (EO)), micro-emulsions (ME), suspension concentrates (SC), aerosols, capsule suspensions (CS) and seed treatment formulations. The formulation type chosen in any instance will depend upon the particular purpose envisaged and the physical, chemical and biological properties of the compound of the present invention.

Dustable powders (DP) may be prepared by mixing a compound of the present invention with one or more solid diluents (for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers) and mechanically grinding the mixture to a fine powder.

Soluble powders (SP) may be prepared by mixing a compound of the present invention with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of the present invention with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of the present invention and one or more powdered solid diluents or carriers, or from pre-formed blank granules by absorbing a compound of the present invention (or a solution thereof, in a suitable agent) in a porous granular material (such as pumice, attapulgite clays, fuller's earth, kieselguhr, diatomaceous earths or ground corn cobs) or by adsorbing a compound of the present invention (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of the present invention in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of the present invention in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of the present invention either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of the present invention is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of the present invention. SCs may be prepared by ball or bead milling the solid compound of the present invention in a suitable medium, optionally with one or more dispersing agents, to produce a fine particle suspension of the compound. One or more wetting agents may be included in the composition and a suspending agent may be included to reduce the rate at which the particles settle. Alternatively, a compound of the present invention may be dry milled and added to water, containing agents hereinbefore described, to produce the desired end product.

Aerosol formulations comprise a compound of the present invention and a suitable propellant (for example n-butane). A compound of the present invention may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of the present invention and, optionally, a carrier or diluent therefor. The polymeric shell may be produced by either an interfacial polycondensation reaction or by a coacervation procedure.

The compositions may provide for controlled release of the compound of the present invention and they may be used for seed treatment. A compound of the present invention may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of the present invention. Such additives include surface active agents (SFAs), spray additives based on oils, for example certain mineral oils or natural plant oils (such as soy bean and rape seed oil), and blends of these with other bio-enhancing adjuvants (ingredients which may aid or modify the action of a compound of the present invention).

Wetting agents, dispersing agents and emulsifying agents may be SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

The compound or composition of the present invention may be applied to a plant, part of the plant, plant organ, plant propagation material or a plant growing locus.

The term “plants” refers to all physical parts of a plant, including seeds, seedlings, saplings, roots, tubers, stems, stalks, foliage, and fruits.

The term “locus” as used herein means fields in or on which plants are growing, or where seeds of cultivated plants are sown, or where seed will be placed into the soil. It includes soil, seeds, and seedlings, as well as established vegetation.

The term “plant propagation material” denotes all generative parts of a plant, for example seeds or vegetative parts of plants such as cuttings and tubers. It includes seeds in the strict sense, as well as roots, fruits, tubers, bulbs, rhizomes, and parts of plants.

The application is generally made by spraying the composition, typically by tractor mounted sprayer for large areas, but other methods such as dusting (for powders), drip or drench can also be used. Alternatively the composition may be applied in furrow or directly to a seed before or at the time of planting.

The compound or composition of the present invention may be applied pre-emergence or post-emergence. Suitably, where the composition is used to regulate the growth of crop plants or enhance the tolerance to abiotic stress, it may be applied post-emergence of the crop. Where the composition is used to promote the germination of seeds, it may be applied pre-emergence.

The present invention envisages application of the compounds or compositions of the invention to plant propagation material prior to, during, or after planting, or any combination of these.

Although active ingredients can be applied to plant propagation material in any physiological state, a common approach is to use seeds in a sufficiently durable state to incur no damage during the treatment process. Typically, seed would have been harvested from the field; removed from the plant; and separated from any cob, stalk, outer husk, and surrounding pulp or other non-seed plant material. Seed would preferably also be biologically stable to the extent that treatment would not cause biological damage to the seed. It is believed that treatment can be applied to seed at any time between seed harvest and sowing of seed including during the sowing process.

Methods for applying or treating active ingredients on to plant propagation material or to the locus of planting are known in the art and include dressing, coating, pelleting and soaking as well as nursery tray application, in furrow application, soil drenching, soil injection, drip irrigation, application through sprinklers or central pivot, or incorporation into soil (broad cast or in band). Alternatively or in addition active ingredients may be applied on a suitable substrate sown together with the plant propagation material.

The rates of application of compounds of the present invention may vary within wide limits and depend on the nature of the soil, the method of application (pre- or post-emergence; seed dressing; application to the seed furrow; no tillage application etc.), the crop plant, the prevailing climatic conditions, and other factors governed by the method of application, the time of application and the target crop. For foliar or drench application, the compounds of the present invention according to the invention are generally applied at a rate of from 1 to 2000 g/ha, especially from 5 to 1000 g/ha. For seed treatment the rate of application is generally between 0.0005 and 150 g per 100 kg of seed.

The compounds and compositions of the present invention may be applied to dicotyledonous or monocotyledonous crops. Crops of useful plants in which the composition according to the invention can be used include perennial and annual crops, such as berry plants for examples blackberries, blueberries, cranberries, raspberries and strawberries; cereals for example barley, maize (corn), millet, oats, rice, rye, sorghum triticale and wheat; fibre plants for example cotton, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

Crops are to be understood as being those which are naturally occurring, obtained by conventional methods of breeding, or obtained by genetic engineering. They include crops which contain so-called output traits (e.g. improved storage stability, higher nutritional value and improved flavour).

Crops are to be understood as also including those crops which have been rendered tolerant to herbicides like bromoxynil or classes of herbicides such as ALS-, EPSPS-, GS-, HPPD- and PPO-inhibitors. An example of a crop that has been rendered tolerant to imidazolinones, e.g. imazamox, by conventional methods of breeding is Clearfield® summer canola. Examples of crops that have been rendered tolerant to herbicides by genetic engineering methods include e.g. glyphosate- and glufosinate-resistant maize varieties commercially available under the trade names RoundupReady®, Herculex I® and LibertyLink®.

Crops are also to be understood as being those which naturally are or have been rendered resistant to harmful insects. This includes plants transformed by the use of recombinant DNA techniques, for example, to be capable of synthesising one or more selectively acting toxins, such as are known, for example, from toxin-producing bacteria. Examples of toxins which can be expressed include δ-endotoxins, vegetative insecticidal proteins (Vip), insecticidal proteins of bacteria colonising nematodes, and toxins produced by scorpions, arachnids, wasps and fungi.

An example of a crop that has been modified to express the Bacillus thuringiensis toxin is the Bt maize KnockOut® (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

Compounds of the present invention may also be used to promote the germination of seeds of non-crop plants, for example as part of an integrated weed control program. A delay in germination of weed seeds may provide a crop seedling with a stronger start by reducing competition with weeds.

Alternatively compounds of the present invention may be used to delay the germination of seeds of crop plants, for example to increase the flexibility of timing of planting for the grower.

Normally, in the management of a crop a grower would use one or more other agronomic chemicals or biologicals in addition to the compound or composition of the present invention. There is also provided a mixture comprising a compound or composition of the present invention, and a further active ingredient.

Examples of agronomic chemicals or biologicals include pesticides, such as acaricides, bactericides, fungicides, herbicides, insecticides, nematicides, plant growth regulators, crop enhancing agents, safeners as well as plant nutrients and plant fertilizers. Examples of suitable mixing partners may be found in the Pesticide Manual, 15th edition (published by the British Crop Protection Council). Such mixtures may be applied to a plant, plant propagation material or plant growing locus either simultaneously (for example as a pre-formulated mixture or a tank mix), or sequentially in a suitable timescale. Co-application of pesticides with the present invention has the added benefit of minimising farmer time spent applying products to crops. The combination may also encompass specific plant traits incorporated into the plant using any means, for example conventional breeding or genetic modification.

The present invention provides the use of a compound of Formula (I), or a composition comprising a compound according to Formula (I) and an agriculturally acceptable formulation adjuvant, for improving the tolerance of a plant to abiotic stress, regulating or improving the growth of a plant, promoting seed germination and/or safening a plant against phytotoxic effects of chemicals.

The present invention also provides the use of a compound, composition or mixture of the present invention, for improving the tolerance of a plant to abiotic stress, regulating or improving the growth of a plant, promoting seed germination and/or safening a plant against phytotoxic effects of chemicals.

There is also provided a seed comprising a compound of Formula (I).

Compounds of Formula (I) may be prepared according to the following general reaction schemes, in which the substituents Y¹, Y², X¹, X², R¹⁹, have (unless explicitly stated otherwise) the definitions described hereinbefore.

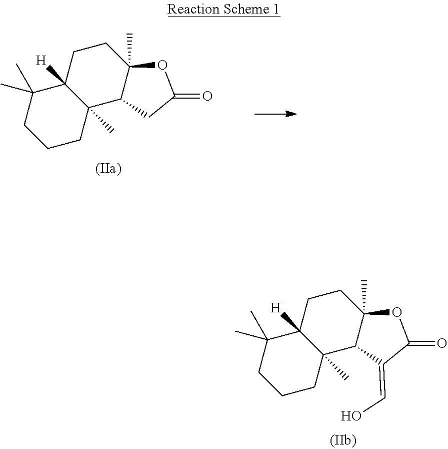 [SMILES: CC1(C)CCC[C@]2(C)[C@H]3CC(=O)O[C@]3(C)CC[C@@H]12 ; CC1(C)CCC[C@]2(C)[C@H]3/C(=C\O)C(=O)O[C@]3(C)CC[C@@H]12]

Known compound of Formula (IIb) (WO2015/061764) may be prepared from commercially available (Sigma-Aldrich) compound of Formula (IIa) via reaction with a formic ester derivative such as the methyl formate in presence of a base such as lithium diidopropylamide, potassium tert-butylate or sodium tert-butylate (WO2012/080115 wherein Y²=NR¹⁸, WO2015/061764 and GB1591374 wherein Y²=O)

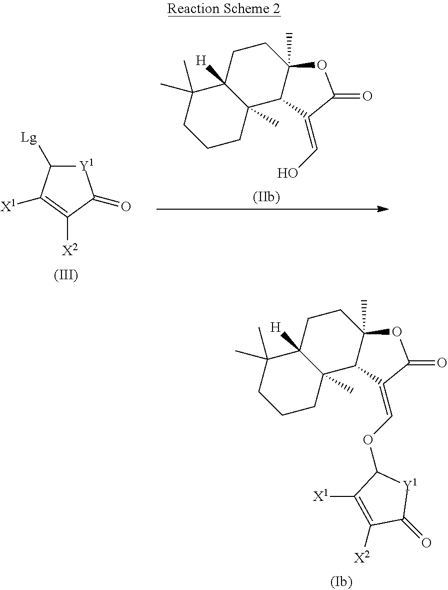

Compounds of Formula (Ib) may be prepared from compounds of Formula (III) by reaction with compound (lib) in the presence of a base such potassium tert-butylate or sodium tert-butylate, and optionally in the presence of a crown ether to activate the base. The reaction can also be carried out in the presence of a catalytic or stoichiometric amount of iodine salt, such as potassium iodide or tetrabutyl ammonium iodide. Compounds of Formula (III) may be prepared from compounds of Formula (IV) or from compounds of Formula (V) as shown in Reaction Scheme 3.

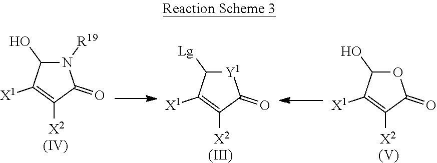

Compounds of Formula (III) wherein Lg is a suitable leaving group, such as halogen, may be prepared from compounds of Formula (IV) or (V) by reaction with a chlorinating agent such as thionyl chloride, phosgene or 1-chloro-N,N,2-trimethyl-1-propenylamine, or a brominating agent such as PBr₃ or thionyl bromide, in the optional presence of a base such as pyridine. Compounds of Formula (III) wherein Lg is a leaving group such alkylsulfonyl or aryl sulfonyl may be prepared from compounds of Formula (IV) by reaction with the corresponding alkylsulfonyl chloride or aryl sulfonyl chloride in the presence of a base such as triethylamine or pyridine. Compounds of Formula (IV) and (V) may be prepared from compounds of Formula (VI) and (VII) respectively as shown in Reaction Scheme 4.

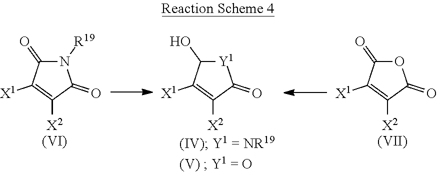

Compounds of Formula (IV) and (V) may be prepared from compounds of Formula (VI) and (VII) respectively by reaction with a reducing agent such as diisopropylaluminium hydride, sodium cyanoborohydride, lithium tri-tert butoxyaluminium hydride or sodium borohydride, optionally in the presence of a Lewis acid such as cerium trichloride. Similar reactions have been reported, for example, in J Chem Soc, Perkin Trans 1, (2002), 707-709 and Journal of Plant Physiology, (2013), 170, 1235-1242. Compounds of Formula (VI) may be prepared from compounds of Formula (VII) as shown in Reaction Scheme 5.

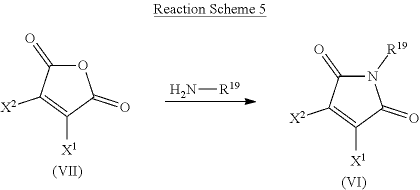

Compounds of Formula (VI) may be prepared from the commercially available compounds of Formula (VII) by reaction with an amine of Formula R¹⁹NH₂ in acetic acid.

### PREPARATION EXAMPLES

The Examples which follow serve to illustrate the invention.

### Compound Synthesis and Characterisation

The following abbreviations are used throughout this section: s=singlet; bs=broad singlet; d=doublet; dd=double doublet; dt=double triplet; bd=broad doublet; t=triplet; dt=double triplet; bt=broad triplet; tt=triple triplet; q=quartet; m=multiplet; Me=methyl; Et=ethyl; Pr=propyl; Bu=butyl; DME=1,2-dimethoxyethane; THF=tetrahydrofuran; M.p.=melting point; RT=retention time, MH⁺=molecular cation (i.e. measured molecular weight).

The following HPLC-MS methods were used for the analysis of the compounds:

Method A: Spectra were recorded on a ZQ Mass Spectrometer from Waters (Single quadrupole mass spectrometer) equipped with an electrospray source (Polarity: positive or negative ions, Capillary: 3.00 kV, Cone: 30.00 V, Extractor: 2.00 V, Source Temperature: 100° C., Desolvation Temperature: 250° C., Cone Gas Flow: 50 L/Hr, Desolvation Gas Flow: 400 L/Hr, Mass range: 100 to 900 Da) and an Acquity UPLC from Waters (Solvent degasser, binary pump, heated column compartment and diode-array detector. Column: Waters UPLC HSS T3, 1.8 μm, 30×2.1 mm, Temp: 60° C., flow rate 0.85 mL/min; DAD Wavelength range (nm): 210 to 500) Solvent Gradient: A=H₂O+5% MeOH+0.05% HCOOH, B=Acetonitrile+0.05% HCOOH) gradient: 0 min 10% B; 0-1.2 min 100% B; 1.2-1.50 min 100% B.

Method B: Spectra were recorded on a ZQ Mass Spectrometer from Waters (Single quadrupole mass spectrometer) equipped with an electrospray source (Polarity: positive or negative ions, Capillary: 3.00 kV, Cone: 30.00 V, Extractor: 2.00 V, Source Temperature: 100° C., Desolvation Temperature: 250° C., Cone Gas Flow: 50 L/Hr, Desolvation Gas Flow: 400 L/Hr, Mass range: 100 to 900 Da) and an Acquity UPLC from Waters (Solvent degasser, binary pump, heated column compartment and diode-array detector. Column: Waters UPLC HSS T3, 1.8 μm, 30×2.1 mm, Temp: 60° C., flow rate 0.85 mL/min; DAD Wavelength range (nm): 210 to 500) Solvent Gradient: A=H₂O+5% MeOH+0.05% HCOOH, B=Acetonitrile+0.05% HCOOH) gradient: 0 min 10% B; 0-2.7 min 100% B; 2.7-3.0 min 100% B.

### Example 1: Preparation of (1E,3aR,5aS,9aS,9bS)-1-(hydroxymethylene)-3a,6,6,9a-tetramethyl-5,5a,7,8,9,9b-hexahydro-4H-benzo[e]benzofuran-2-one (Compound IIb)

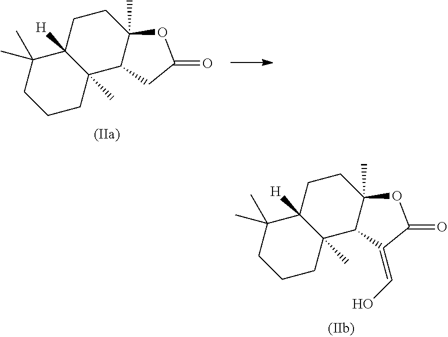 [SMILES: CC1(C)CCC[C@]2(C)[C@H]3CC(=O)O[C@]3(C)CC[C@@H]12 ; CC1(C)CCC[C@]2(C)[C@H]3/C(=C\O)C(=O)O[C@]3(C)CC[C@@H]12]

(1E,3aR,5aS,9aS,9bS)-1-(hydroxymethylene)-3a,6,6,9a-tetramethyl-5,5a,7,8,9,9b-hexahydro-4H-benzo[e]benzofuran-2-one (compound of Formula (IIb)) was prepared from commercially available (Sigma-Aldrich) compound (IIa) as described in WO2015/061764. ¹H NMR (400 MHz, CDCl₃): δ ppm 9.58 (d, 1H), 3.59 (dd, 1H), 2.49 (d, 1H), 1.18 (dt, 1H), 1.94 (m, 1H), 1.79 (dt, 1H), 1.56-1.72 (m, 1H), 1.32-1.51 (m, 6H), 1.09-1.26 (m, 4H), 0.97 (bs, 3H), 0.90 (bs, 3H), 0.83 (bs, 3H).

### Example 2: Preparation of 1-(phenyl)-3,4-dimethyl-pyrrole-2,5-dione (Compound VI-1)

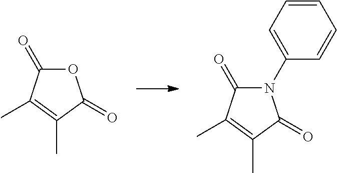

1-(phenyl)-3,4-dimethyl-pyrrole-2,5-dione (VI-1) was prepared following a slightly modified reported procedure (J. Org. Chem. 1998, 63, 2646-2655). To a solution of 2,3-dimethylmaleic anhydride (118.9 mmol, 15 g) in acetic acid (200 mL) was added aniline (120 mmol, 11.0 mL) and the resulting suspension was heated at 132° C. for 24 hours. The reaction mixture was then cooled to room temperature, the solvent removed under reduced pressure and the resulting crude residue was purified by flash chromatography over silica. 1-(phenyl)-3,4-dimethyl-pyrrole-2,5-dione (VI-1) was isolated as a white solid (18.0 g, 89.5 mmol, 75% Yield). LCMS (method A): RT 0.86 min; ES+202 (M+H⁺); ¹H NMR (400 MHz, CDCl₃): δ ppm 2.07 (s, 6H), 7.31-7.41 (m, 3H), 7.42-7.53 (m, 2H).

### Example 3: 2-hydroxy-3,4-dimethyl-1-(phenyl)-2H-pyrrol-5-one (compound IV-1)

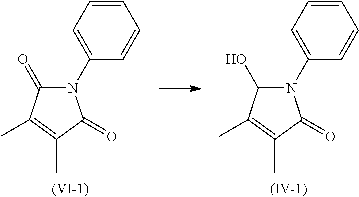

1-(phenyl)-3,4-dimethyl-pyrrole-2,5-dione (compound VI-1, 84.5 mmol, 17 g) was dissolved in methanol (84 mL) and cooled to 0° C. Sodium borohydride (0.486 g, 12.6 mmol) was added portion wise and the mixture was stirred for 2 hours. Ice water was added slowly and methanol was removed under reduced pressure. The crude product was taken up in water, diluted with ethyl acetate and the phases separated. The organic fraction was washed with brine, dried over sodium sulfate and concentrated under vacuum. 2-hydroxy-3,4-dimethyl-1-(phenyl)-2H-pyrrol-5-one (IV-1) was isolated as a pure pink solid and used without further purification. LCMS (method B): RT 0.82 min; ES− 202 (M−H⁺); ¹H NMR (CDCl₃, 400 MHz): δ ppm 1.50 (m, 3H), 1.98 (s, 3H), 5.56 (bs, 1H), 7.10 (m, 1H), 7.31 (m, 2H), 7.70 (m, 2H).

### Example 4: 2-chloro-3,4-dimethyl-1-(phenyl)-2H-pyrrol-5-one (compound III-1)

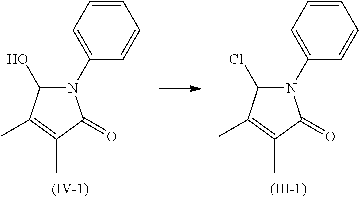

To a solution of 2-hydroxy-3,4-dimethyl-1-(phenyl)-2H-pyrrol-1-one (IV-1, 2.1 mmol, 5.50 g) in dichloromethane (140 mL) under argon was added 1-chloro-N,N,2-trimethyl-1-propenylamine (32.5 mmol, 4.48 mL). The reaction mixture was stirred at room temperature for 72 hours and concentrated in vacuo to give an oil containing the desired product in mixture with N,N-2-trimethylpropanamide. 2-chloro-3,4-dimethyl-1-(phenyl)-2H-pyrrol-5-one (compound III-1, 26.5 mmol, 5.88 g, 98% yield) was used as such for the next step. ¹H NMR (400 MHz, CDCl₃): δ ppm 1.95 (s, 3H), 2.15 (s, 3H), 6.18 (s, 1H), 7.15-7.26 (t, 1H), 7.35-7.48 (t, 2H), 7.56-7.68 (d, 2H).

### Example 5: 2-[(E)-[(3aR,5aS,9aR,9bS)-3a,5a,6,6,9a-pentamethyl-2-oxo-4,5,7,8,9,9b-hexahydrobenzo[e]benzofuran-1-ylidene]methoxy]-3,4-dimethyl-1-phenyl-2H-pyrrol-5-one (compound Ib-1)

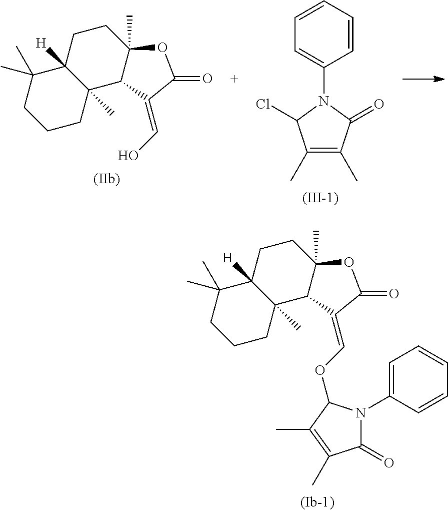

A solution of compound (11) (0.60 g, 1.94 mmol) in 1,2-dimethoxyethane (20 mL) under argon was cooled to 0° C. and potassium tert-butylate (0.24 g, 2.13 mmol) was added. After stirring for 5 minutes at 0° C., compound (111-1) (0.49 g, 2.2 mmol) in 1,2-dimethoxyethane (5 mL) was added and the reaction mixture was stirred at room temperature for 16 h. Aqueous NH₄Cl solution and ethyl acetate were added, and the aqueous layer extracted with ethyl acetate. The combined organic layers were washed with brine, dried over Na₂SO₄, and the solvent removed in vacuo. The residue was purified by flash column chromatography over silica to give Ib-1 as a white solid as a mixture of diastereomers (0.22 g, 0.47 mmol, 24% yield); LCMS (Method B): RT 2.16 min; ES− 462 (M−H+); ¹H NMR (400 MHz, CDCl₃) for one diastereomer: δ ppm 0.75-2.08 (m, 29H), 2.47 (d, 1H), 6.07 (bs, 1H), 6.20 (d, 1H), 7.14 (t, 1H), 7.36 (t, 2H), 7.66 (m, 2H).

### Example 6: (1E,3aR,5aS,9aR,9bS)-1-[(3,4-dimethyl-5-oxo-2H-furan-2-yl)oxymethylene]-3a,5a,6,6,9a-pentamethyl-4,5,7,8,9,9b-hexahydrobenzo[e]benzofuran-2-one (compound Ib-19)

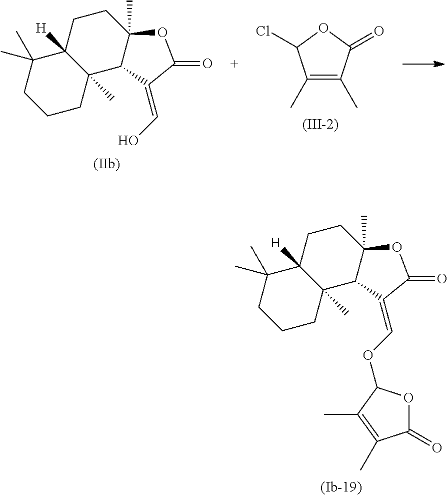

Compound (Ib-19) may be prepared according to a similar procedure as that utilized in the synthesis of compound (Ib-1) using known compound (III-2) (WO2012/056113). Compound (Ib-19) was isolated as a mixture of diastereoisomers. LCMS (Method A): RT 1.14 min; ES+375 (M+H⁺); ¹H NMR (400 MHz, CDCl₃) for one diastereomer: δ ppm 0.78-2.23 (m, 29H), 2.53 (m, 1H), 5.91 (bs, 1H), 7.36 (d, 1H).

### BIOLOGICAL EXAMPLES

### Example 1: Corn Seed Germination

The effect of compounds of Formula (I) on the germination of NK Falkone corn seeds under cold stress was evaluated as follows.

NK Falkone corn seeds were sorted by size using 2 sieves, one excluding very big seeds and the other with round holes of 8 to 9 mm diameter. The seeds retained by the latter sieve were used for the germination test.

The corn seeds were placed in 24 well plates (each plate was considered as one experimental unit or replicate). Germination was initiated by the addition of 250 μl of distilled water containing 0.5% DMSO per well as a mean for compound solubilization. 8 replicates (ie, 8 plates) were used for each treatment characterization. Plates were sealed using seal foil (Polyolefin Art. Nr. 900320) from HJ-BIOANALYTIK. All plates were placed horizontally on trolleys in a climatic chamber at 15° C. or 23° C. in complete darkness. The experiment was laid out in a completely randomized design in climatic chamber with 75% Relative Humidity. Foils were pierced, one hole per well using a syringe after 72 hours for experiments performed at 15° C. and after 24 hours for experiments performed at 23° C.

GR24 is a commercially available strigolactone analogue.

AB01 was disclosed in WO 2015/061764; it is a chemical mimic of strigolactone where X¹ is hydrogen, and is therefore a close analogue to compounds of the present invention.

Germination was followed over time by taking photographs at different time points. Image analysis was performed automatically with a macro which was developed using the Image J software. A dynamic analysis of germination was carried out by fitting a logistic curve. Three parameters were calculated from the logistic curve: the T50; the slope and the plateau. All three parameters have a high agronomical relevance and are key requirements to ensure a good early crop-establishment. The T50, slope and plateau for a selection of compounds are outlined in Table 2 below. All the values are expressed as percentages compared to an untreated control. All the three parameters are calculated considering 8 replicates and the kinetic parameters are separately determined for each germination curve. Data in bold indicate germination enhancing statistically significant differences between treated seeds and untreated control (p<0.05).
T50 corresponds to the time needed for half of the seed population to germinate. Higher negative %-values indicate faster germination.
Slope indicates how synchronous the germination of the seed population is. Positive values indicate steeper curve. The steeper the curve, the better and more uniform the germination is.
Plateau provides information about the final germination rate and it is expressed in percentage.

Positive values indicate a larger number of seeds germinated in a given period.

TABLE 2
Effect of strigolactone analogues on germination of corn seeds
under cold stress condition (15° C.) at various concentrations.

| Com- | Rate | Plateau(% | Slope (% | T50 (% |
|---|---|---|---|---|
| pound | (μM)^{a} | vs control)^{b} | vs control)^{b} | vs control)^{b} |
| GR-24 | 0.08 | 2.10 | −8.50 | −0.10 |
| | 0.4 | 0.90 | 0.20 | −1.80 |
| | 2 | −1.40 | −0.80 | −0.50 |
| | 10 | 1.50 | 7.40 | −2.40 |
| AB01 | 0.04 | −2.20 | 6.50 | −1.80 |
| | 0.2 | −2.20 | 10.00 | 0.50 |
| | 1 | 3.00 | 27.50 | −2.30 |
| | 5 | 1.30 | 16.50 | −1.90 |
| Ib-1 | 0.08 | 6.50 | −1.40 | −4.40 |
| | 0.4 | 4.70 | −10.00 | 3.20 |
| | 2 | 4.70 | 11.20 | −0.60 |
| | 10 | 7.10 | 62.00 | −7.90 |
| Ib-19 | 0.04 | 7.10 | 13.60 | −1.10 |
| | 0.2 | −4.60 | 11.40 | −3.20 |
| | 1 | 3.00 | 7.60 | 0.20 |
| | 5 | 8.30 | 24.20 | −2.80 |

^{a}Concentration in compound (I) in 250 μl distilled water containing 0.5% DMSO
^{b}Control = 250 μl distilled water containing 0.5% DMSO

The results show that seeds tested with compounds of the present invention result in better germination of corn seed under cold stress than the standards.

### Example 2: Hydrolytic Stability Assay

The objective of the hydrolytic stability assay was to determine the chemical stability of the individual test compounds in a strictly controlled and reproducible environment allowing a comparison of their in-vitro stability under aqueous conditions at pH7 and 9.

Due to low solubility of these analogues, a percentage of acentonitrile is added to the system to aid solubility (nominally 10-50%). Prior to conducting the individual assays, 1000 ppm stock solutions of all four test compounds were prepared in methanol. The reagents used in the assays were prepared as follows:

20 mM buffer solution: A stock of 20 mM mixed acetate, borate and phosphate buffer was prepared and the pH adjusted to 7 or 9 as required.

Test solutions were prepared in LC vials for each test compound in the following manner: Mobile Phase Control: Mobile phase (1 ml)+compound (0.5-40 μl);

Hydrolytic Stability: Buffer (1 ml)+compound (0.5-40 μl).

The mobile phase and buffer were dispensed into separate glass LC vials, placed into a thermostatted autosampler set at 40° C., and allowed to equilibrate for 30 minutes prior to starting the individual assays.

Reactions were initiated by addition of compound and monitored through a series of repeat injections made directly from the vial into an HPLC system at regular time intervals.

Initial and subsequent measurements of peak area attributable to the test compound were used to fit exponential half-lives and calculate first-order rate constants.

Definitive half-lives could not be determined for test compounds Ib-19 and AB01 at pH7, and for compound Ib-1 at pH7 and pH9, as insufficient loss was observed under the experimental conditions employed. Consequently, the percent remaining was recorded at the last assessment time.

Stability data (t1/2 meaning the time in hours for half of the test compound to be hydrolysed), are provided in Table 3 below.

TABLE 3
Hydrolytic stability of compounds of the present invention (Ib-1 and Ib-19)
(disubstituted butenolid) versus prior art compound AB01 (monomethyl butenolide)

| | Hydrolytic Stability (t_{1/2}, hours) | |
|---|---|---|
| Compound | pH 7 + 25% MeCN | pH 9 + 25% MeCN |
| 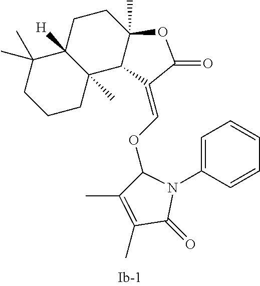 [SMILES: CC1=C(C)C(O/C=C2/C(=O)O[C@]3(C)CC[C@H]4C(C)(C)CCC[C@]4(C)[C@@H]23)N(c2ccccc2)C1=O] | >18.1^{a} | >17.8^{c} |
| 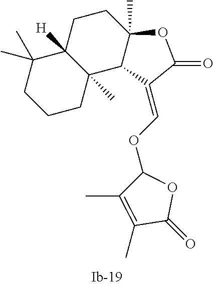 [SMILES: CC1=C(C)C(O/C=C2/C(=O)O[C@]3(C)CC[C@H]4C(C)(C)CCC[C@]4(C)[C@@H]23)OC1=O] | >17.6^{a} | 19.4 |
| 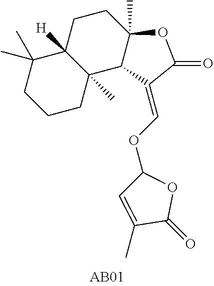 [SMILES: CC1=CC(O/C=C2/C(=O)O[C@]3(C)CC[C@H]4C(C)(C)CCC[C@]4(C)[C@@H]23)OC1=O] | >17.6^{b} | 3.0 |

^{a}100% remaining at final timepoint
^{b}96% remaining at final timepoint
^{c}97% remaining at final timepoint

The results show that compounds (Ib-1) and (Ib-19) of the present invention have superior hydrolytic stability to the prior art compound at the biologically-relevant pH levels of pH9.

### Example 3: Soil Stability Assay

It is highly desirable that agrochemicals applied to soil in order to deliver a beneficial biological effect can exist in the soil for a prolonged period of time with minimal degradation. However, a biologically active agrochemical compound may undergo chemical transformation in soil, leading to decreased levels of activity and a decrease in a desired biological effect. Simple laboratory degradation studies can be used to evaluate the disappearance due to biotic and abiotic processes of a compound in soil. The time taken for a compound to degrade in soil allows the estimation of their half-life (t1/2), which corresponds to the time in which 50% of the compound under evaluation is degraded in soil. This can be a useful parameter to evaluate the stability of a compound in soil, with the longer the half-life, the more stable the compound.

### Sample Preparations

### Standard Solutions/Treatment Solution

Stock standard solutions were prepared by dissolving 1 mg of each test compound (ie, compounds (Ib-1, Ib-19 and AB01) in acetonitrile. The stock standard solutions were stored at 6° C. Working standard solutions were then obtained by a series of dilutions of the stock standard solutions for an external calibration. A treatment solution of 100 μg/mL concentration for each test compound was prepared in acetonitrile:water (6:4)(v:v).

### Soil Preparation

Soil samples used for this soil stability assay were collected at the Syngenta Research Centre location in Stein (Switzerland). The soil was classified as clay loam soil. Certain physical properties of the soil are described in Table 4.

TABLE 4
Physical properties of Stein soil

| | | | | | | Cation | Water Hold | Water Hold |
|---|---|---|---|---|---|---|---|---|
| | | | | | Organic | Exchange | Capacity at | Capacity at |
| Water | CaCl₂ | Sand | Silt | Clay | Matter | Capacity | 0.33 bar | 15 bar |
| pH | pH | % | % | % | % | M eq/100 g | % | % |
| 7.9 | 7.4 | 30 | 43 | 27 | 3.5 | 19.4 | 25.8 | 14.8 |

2 mm sieved Stein soil was mixed with sand at ratio 1:1 prior to starting the laboratory soil degradation experiment. 10 g of the sand-soil mix (air-dried basis) was weighed into 50 ml Corning® polypropylene centrifuge tubes and soil moisture was adjusted at 45% of the field capacity.

### Chemical Application and Incubation Conditions

Chemical application was performed by applying 30 μl of a 100 μg/mL solution of each test compound to 10 g soil vessel corresponding to a final concentration of 0.3 μg test compound per gram of soil. Three replicates were considered for each test compound. Treated tubes were incubated in the dark at 20° C.±0.5 with 85% relative humidity. For the estimation of half-life, different sampling times of 0, 4, 8, 24, 72, 168 and 336 hours were considered. At each sampling time, samples were removed and stored at −80° C. until analysis. The half-lives were calculated by an exponential regression analysis (single first order kinetic model) plotting the percentage of recovered compound in soil against the time.

### Chemical Extraction and Analysis

Compounds AB01, Ib-1 and Ib-19 were extracted from soil by using 30 mL of Acetonitrile (CHROMASOLV® gradient grade, for HPLC, >99.9%, SIGMA-ALDRICH). The mixture was shaken for 3 hours at room temperature by using a vertical rotary shaker. After centrifugation at 3500 rpm for 5 minutes, an aliquot of the supernatant was collected and analyzed via UPLC-MS (Waters Acquity UPLC-MS PDA-Detection: 254 nm- and SQD-Zspray ESI, APCI, ESCi®-; Waters Acquity UPLC Column HSS T3 2.1×30 mm-1.8 μm; Gradient mobile phase with H2O:MeOH (9:1, v:v)+0.1% HCOOH (solvent A) and MeCN+0.1% HCOOH (solvent B); 30% to 100% of solvent B in 1 min, then 100% of solvent B for 0.45 min and then down to 30% solvent B at 1.5 min.; flow rate 0.75 mL·min−1). The results are shown in Table 5.

TABLE 5
Soil stability of compounds of the present invention (Ib-1
and Ib-19) (disubstituted butenolide) versus prior
art compound AB01 (monomethyl butenolide)

| | Soil Stability |
|---|---|
| Compound | (t_{1/2}, hours) |
| 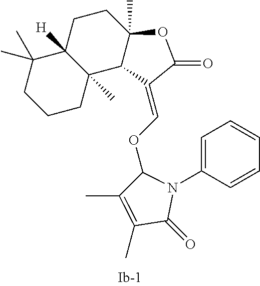 [SMILES: CC1=C(C)C(O/C=C2/C(=O)O[C@]3(C)CC[C@H]4C(C)(C)CCC[C@]4(C)[C@@H]23)N(c2ccccc2)C1=O] | >720 |
| 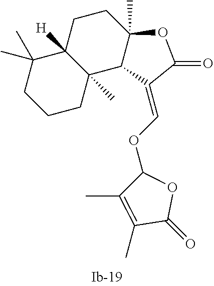 [SMILES: CC1=C(C)C(O/C=C2/C(=O)O[C@]3(C)CC[C@H]4C(C)(C)CCC[C@]4(C)[C@@H]23)OC1=O] | 280 |
| 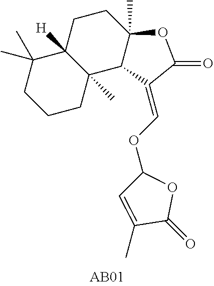 [SMILES: CC1=CC(O/C=C2/C(=O)O[C@]3(C)CC[C@H]4C(C)(C)CCC[C@]4(C)[C@@H]23)OC1=O] | 38 |

The results show that compound (Ib-1) and (Ib-19) of the present invention exhibit superior soil stability compared to prior art compound AB01.

1. A compound of Formula (I) 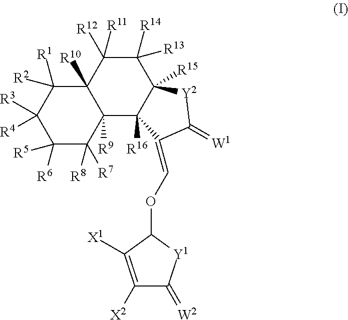
wherein
R¹, R², R³, R⁴, R⁵, R⁶, R⁷, R⁸, R⁹, R¹⁰, R¹¹, R¹², R¹³, R¹⁴, R¹⁵ and R¹⁶ are each independently hydrogen, C₁-C₆alkyl, C₁-C₆haloalkyl, halogen, OR¹⁷, cyano, or N(R¹⁸)₂, wherein R¹⁸ may the same or different;
R¹⁷ is hydrogen, C₁-C₆alkyl, C₁-C₆haloalkyl, C₂-C₆alkenyl, C₂-C₆alkynyl, C₃-C₆cycloalkyl, C₁-C₈alkylcarbonyl, C₁-C₈alkoxycarbonyl, substituted or unsubstituted aryl, substituted or unsubstituted heteroaryl, substituted or unsubstituted heterocyclyl, substituted or unsubstituted benzyl;
R¹⁸ is hydrogen, C₁-C₆alkyl, C₁-C₆alkoxy, C₃-C₆cycloalkyl, C₂-C₆alkenyl, C₂-C₆alkynyl, C₁-C₅alkylcarbonyl, C₁-C₅alkoxycarbonyl, hydroxyl, amino, N—C₁-C₆alkylamine, N,N-di-C₁₋C₆alkylamine, substituted or unsubstituted aryl, substituted or unsubstituted heteroaryl, substituted or unsubstituted heterocyclyl, substituted or unsubstituted benzyl;
W¹ and W² are independently oxygen or sulfur;
Y¹ and Y² are independently oxygen, sulfur, or NR¹⁹;
R¹⁹ is hydrogen, C₁-C₆alkyl, C₁-C₆haloalkyl, C₁-C₆alkoxy, C₃-C₆cycloalkyl, C₂-C₆alkenyl, C₂-C₆alkynyl, C₁-C₈alkylcarbonyl, C₁-C₈alkoxycarbonyl, hydroxyl, amine, N—C₁-C₆alkylamine, N,N-di-C₁-C₆alkylamine, substituted or unsubstituted aryl, substituted or unsubstituted heteroaryl, substituted or unsubstituted heterocyclyl, substituted or unsubstituted benzyl; and
X¹ is selected from C₁-C₆alkyl, C₂-C₅₃alkynyl, C₁-C₆haloalkyl, halogen, hydroxyl, C₁-C₆alkoxy, C₁-C₆alkylsulfinyl, C₁-C₆alkylsulfonyl, C₁-C₆alkylthio, OR¹⁷ and N(R¹⁸)₂;
X² is selected from hydrogen, C₁-C₆alkyl, C₂-C₃alkynyl, C₁-C₆haloalkyl, halogen, hydroxyl, C₁-C₆alkoxy, C₁-C₆alkylsulfinyl, C₁-C₆alkylsulfonyl, C₁-C₆alkylthio, OR¹⁷ and N(R¹⁸)₂; or
X¹ and X² together with the carbon atoms to which they are attached form a C₅- or C₆-cycloalkyl;
or salts or N-oxides thereof.

2. The compound according to claim 1 wherein Y¹ is oxygen.

3. The compound according to claim 1 wherein Y¹ is N(R¹⁹).

4. The compound according to claim 1 wherein X¹ and X² are independently C₁-C₆alkyl, C₁-C₆alkoxy or C₁-C₆haloalkyl.

5. The compound according to claim 4 wherein X¹ and X² are independently selected from methyl, ethyl and methoxy.

6. The compound according to claim 5 wherein X¹ and X² are both methyl.

7. The compound according to claim 1 wherein R¹⁹ is phenyl or phenyl substituted by one to five R²⁰.

8. The compound according to claim 7 wherein R²⁰ is C₁-C₄haloalkyl.

9. The compound according to claim 1, as defined by Formula (Ia):

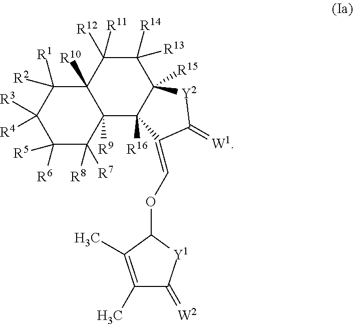

10. The compound according to claim 1, as defined by Formula (Ib):

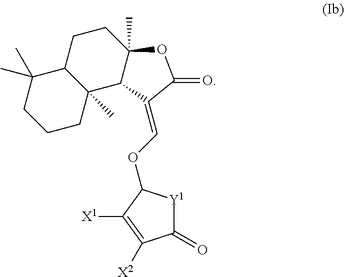

11. A plant growth regulating or seed germination promoting composition, comprising the compound according to claim 1, and an agriculturally acceptable formulation adjuvant

12. A mixture comprising a compound according to claim 1 and a further active ingredient.

13. A method for regulating the growth of plants, said method comprising applying to the plants or a locus containing the plants a compound according to claim 1.

14. A method for promoting the germination of seeds, comprising applying to the seeds, or a locus containing the seeds, the compound according to claim 1.

15. (canceled)

16. A seed comprising a compound of Formula (I) according to claim 1.
